(19) United States
(12) Patent Application Publication
(10) Pub. No.: US 2009/0000618 A1
(43) Pub. Date: Jan. 1, 2009

(54) SINGLE SIDED HOUSING FOR MEDICAL CANULA TUBING COMBINING WIRELESS CELLULAR PHONE AND AUDIO TECHNOLOGY WITH OXYGEN DELIVERY SYSTEMS

(76) Inventor: Sydney Warren, Marina del Rey, CA (US)

Correspondence Address: BELASCO, JACOBS & TOWNSLEY LLP;HOWARD HUGHES CENTER, 6100 CENTER DRIVE, SUITE 630, LOS ANGELES, CA, 90045, US

(21) Appl. No.: 12/204,682

(22) Filed: Sep. 4, 2008

Related U.S. Application Data
(63) Continuation-in-part of application No. 12/131,746, filed on Jun. 2, 2008.
Application No. 12/131,746 is a division of application No. 10/945,546, filed on Sep. 20, 2004.

(51) Int. Cl. A61M 16/00

(52) U.S. Cl. 128/202.13

(57) ABSTRACT

A portable gas delivery system includes a mouthpiece or nosepiece, a gas delivery pack and a medical cannula tubing assembly. The mouthpiece has an upper region and a lower region. The upper region includes at least one nose port. The lower region includes a mouth port. The nosepiece conceals the cannula and includes at least one nose port. The gas delivery pack has a gas storage region, a gas compression region, a gas filtration region or a combination thereof. The nosepiece, allows the cannula to provide a snug fit into the nostrils forming a medical airtight delivery of oxygen to the nose. The system is adapted for use with a wireless transceiver for use with a cellular telephone or a personal music system and connected headphones. A single-sided, hollow frame conceals the mouthpiece or nosepiece and tubing assembly while attaching to a hat, eyeglass frame or ear of a user.

### REFERENCE TO RELATED APPLICATIONS

The present application is a continuation-in-part of U.S. divisional application Ser. No. 12/131,746, filed Jun. 2, 2008, which is a divisional application of U.S. application Ser. No. 10/945,546 filed Sep. 20, 2004.

### FIELD OF THE INVENTION

The invention relates generally to a system for delivering oxygen and/or air. More particularly, the invention relates to a portable system for delivering oxygen and/or air to persons in a comfortable and convenient manner while using personal telecommunications and audio devices during daily activities.

### BACKGROUND OF THE INVENTION

Clean air is an important part of maintaining health in an otherwise healthy person. Environments that contain air-borne pollutants and infectious agents have received much publicity and awareness. Second hand cigarette smoke, carbon monoxide, SARS, dust, pollen and car exhaust, among other such pollutants can cause respiratory discomfort, damage or inefficiency.

Some of these effects can be temporary, as when exposed to an allergen where the effect disappears when the allergen is removed, and some of these effects can be permanent, as when second-hand cigarette smoke causes cancer. It is beneficial to an otherwise healthy person to maintain a high quality supply of breathable air by either filtering the ambient air before inhalation, or providing an alternate source for the breathable air that excludes a large fraction of the ambient air.

This effect is well known for unhealthy people, such as an emphysema patient who pulls a cart with an oxygen tank that supplies oxygen to an area around the nose or mouth to enhance the oxygen uptake of damaged lungs. Another example is the oxygen masks used in hospitals to provide a similar effect for the same or other medical purpose. These systems provide higher quality air in a manner that is not easily portable for an individual, and certainly not designed to be fashionable or attractive.

A personal oxygen and/or air delivery system must further meet certain functional requirements. It must be able to remove some potentially, perceived or actually harmful fraction of the ambient air, or must be able to provide at least a portion of the inhaled air by displacing at least a portion, if not all, of the ambient air with either air filtered by an air filtration pump, or air from a compressed gas tank of appropriate dimension so as to be worn on the body. In the alternative it may be required to do both functions, with some portion of the inhaled air coming through the filter, some portion coming from the gas tank, and the remaining air, if any, coming from the ambient air.

For the purposes of this application, higher quality air refers to air with either more desirable characteristics, air which has fewer undesirable characteristics, or air which has both more desirable and fewer undesirable characteristics. Percent O₂ content, pollen, particles, aromatic compounds, gaseous and particulate carbon compounds including hydrocarbons, nitrogen containing compounds, carbon monoxide, ozone, viral infectious agents, bacterial infectious agents, sulfur containing compounds, dust, soot, smoke, smog, and many other compounds can, among many other factors, be factors used when determining characteristics of air quality. Desirable and undesirable characteristics can be left to the objective determination of government and industry agencies or subjective determination of each individual user of this system.

As air quality in some cities declines, and as the perception that the number and quantity of harmful components in the air is increasing, there is a growing group of people who wish to breath higher quality air but are unwilling to pull a tank of higher quality air around in a cart, or unwilling to wear a standard mask or nose-piece normally associated with an unhealthy person, and thus convey a negative body image to others or to themselves.

Further, there has developed a desire by some people to stop periodically in commercial locations called oxygen bars, or commercial locations, like health clubs, that offer oxygen bars in addition to other services. These commercial locations provide customers with, among other services, an opportunity to temporarily inhale breathable air that can contain higher concentrations of oxygen.

Such breathable air may also be enhanced with aromas pleasing to the customer. Currently these customers use a small flexible hose called a cannula to deliver the oxygen-enriched breathable air into their nostrils. As part of the experience of being in an oxygen bar and social setting, it is desirable to portray the experience as glamorous or fashionable in addition to being functional.

Accordingly, it is desirable for the cannula to be incorporated into other devices normally appearing on or around the face of a person to enhance the appearance of glamour or fashion, or to disguise or otherwise hide the functional aspect of the cannula.

Various embodiments of the present invention are directed to addressing various needs in connection with ensuring that higher quality air is delivered to the nose and or mouth area of a person wearing the system disclosed in this invention.

As people feel the need to maintain communications in a near constant manner, they often make use of portable wireless earpieces. In a similar manner, many people choose to take advantage of compact headphones for use with personal music systems. The combination of these devices with oxygen/air breathing devices represents new flexibility and freedom for those who desire to take advantage of both systems in a comfortable and convenient manner.

Various patents address systems that are designed to permit the wearer to breathe oxygen or purified air. Sanders, U.S. Pat. No. 5,582,164, discloses a portable gas delivery system that includes a gas storage assembly that is connected to a nose piece using flexible tubing. The gas storage assembly includes a strap that enables it to be readily carried by the person using this system.

AmRhein, U.S. Pat. No. 4,996,983, discusses a portable oxygen delivery system in which gas storage containers are incorporated into the temples on eyeglasses. Air delivery tubes extend around the wearer's ears to a nose plug that is placed in the wearer's nose.

Peppler, U.S. Pat. No. 5,193,534; and Timmons et al., U.S. Pat. No. 4,559,941, both describe incorporating an oxygen delivery system into eyeglasses. The oxygen is delivered from a storage container to the temples on the eyeglasses. The oxygen is then routed to a nosepiece through the eyeglasses.

Koch et al., U.S. Pat. No. 4,465,067, discloses a system for delivering oxygen. This system has a configuration that is similar to eyeglasses except that it does not include any lenses. Oxygen is delivered to the temple portion that wraps around the wearer's ear and then to a nose piece that is positioned proximate the wearer's nose.

Izuchukwu et al., U.S. Pat. No. 6,526,968, discusses an air delivery system that is incorporated into a utility belt that is worn by the user. The utility belt includes a pack for storing the compressed gas and a port for connecting to a mask using flexible tubing.

Izuchukwu et al., U.S. Pat. No. 6,510,859, discloses an emergency breathing apparatus that includes a hood, which is placed over the user's entire head. Air is delivered to the hood from a storage pack. While such a system is acceptable for emergency situations, the fact that it covers the user's entire head limits the ability for the user to perform many activities while wearing this device.

McDonald, et al., U.S. Pat. No. 6,595,207, describes an oxygen diffuser for a lightweight oxygen delivery system for a patient, the oxygen delivery system being of the type comprising a mount for seated engagement on a patient's head or ear, an elongated tubular boom for oxygen delivery secured at one end to the mount and having the diffuser secured to the other end, the diffuser to deliver oxygen passed through the boom to a space in the vicinity of the patient's nose and mouth, the diffuser comprising a body having a wall, the interior surface of which wall is of generally concave configuration, circumscribing a centrally positioned oxygen outlet so as to direct the flow of oxygen from the outlet generally towards the patient's nose and mouth; and a baffle seated over the oxygen outlet so as to assist in mixing of oxygen with ambient air and avoid a direct flow of oxygen towards the patient's face.

Demers, et al., U.S. Pat. No. 6,619,288, describes a breathing mask for delivering oxygen to a patient. The breathing mask has a headset for seating about the cranial region of the head of the patient, with a hollow gas delivery arm coupled to the headset. The mask also has a nosepiece that is coupled only to the hollow gas delivery arm and that delivers oxygen to the nostrils of the patient. Finally, the mask has a source of oxygen for coupling oxygen to the hollow gas delivery arm.

McCombs, et al., U.S. Pat. No. 6,065,473, describes a non-contact gas dispenser comprising a head set apparatus, a gas source and a conduit for directing the desired gas to a region proximate to the user's nose and mouth. The gas source preferably is a pressure swing adsorption apparatus that allows the user to select one of at least two pre-determined settings of product gas, each setting having a distinct concentration and flow rate different from the other setting(s).

Martin, James F. et al., U.S. Patent Application No. 2006/0042631, describes an oxygen delivery device for regulating the flow of oxygen from an oxygen source to a respiratory cannula located on the face of a patient. A host controller coupled to the oxygen delivery device accepts an input from a medical monitor relating to the blood oxygen saturation percentage of the patient. The host controller then regulates the rate of oxygen supplied to the patient based in part upon the blood oxygen saturation percentage of the patient. A high rate of oxygen is supplied to the patient when the controller detects a blood oxygen saturation level below a predetermined percentage and a low rate of oxygen is supplied when the controller detects a blood oxygen saturation level above a predetermined percentage in an order to minimize wasted oxygen.

It is an objective of the present invention to provide a personal oxygen/air breathing device that is attractive, comfortable and convenient for daily wear. It is a further objective to provide such a system that can be combined with hats, masks, visors, eyeglasses, sunglasses and other fashion accessories. It is a still further objective to provide a single-sided housing in the shape of a ‘boom’ to shield and conceal standard medical cannula tubing and technology. It is another objective to utilize the same medical cannula tubing within the housing universally used in all hospitals around the world supplying an ‘air-tight’ delivery of oxygen to the user's nose. It is yet another objective to provide such breathing gear that is combined with a wireless personal earpiece for use with a cellular telephone. Finally, it is an objective of the present invention to provide breathing systems that are combined with personal headphones for use with portable music players.

### SUMMARY OF THE INVENTION

(1) A portable gas delivery system is constructed from the following components. A mouthpiece is provided that has an upper region and a lower region extending from it. The upper region includes at least one nose port and the lower region includes a mouth port. A gas delivery pack is provided that has a gas storage region, a gas compression region, a gas filtration region or a combination thereof. A medical cannula tubing assembly is provided that is operably attached to the mouthpiece and the gas delivery pack for delivering gas from the gas delivery pack to the mouthpiece. A single-sided, hollow frame is provided. The frame at least partially encloses the mouthpiece and the tubing assembly and is adapted to attach to an ear, a hat, helmet or an eyeglass frame of a user. A wireless transceiver adapted for use with a cellular telephone is provided. The transceiver has a listening portion and a speaking portion and is attached to the frame assembly so as to position the listening portion adjacent a user's ear and the speaking portion adjacent a user's mouth.

(2) In a variant of the invention, the mouthpiece includes a switch for controlling flow of oxygen and air through the mouthpiece.

(3) In another variant, the at least one nose port is capable of forming a medical standard air-tight seal when inserted into a nostril.

(4) In still another variant, the mouth port is capable of forming a substantially air-tight seal when inserted into a mouth.

(5) In yet another variant, the tubing assembly includes a least one rotatable connector adjacent the listening portion.

(6) In a further variant, includes at least one adapter that has a first recess and a second recess formed in it. The first recess is adapted to receive a portion of the tubing assembly and the second recess is adapted to receive a strap for mounting the tubing assembly with respect to a person's head.

(7) In still a further variant, a portable gas delivery system includes a mouthpiece that has an upper region and a lower region extending from it. The upper region includes at least one nose port and the lower region includes a mouth port. A gas delivery pack is provided that has a gas storage region, a gas compression region, a gas filtration region or a combination thereof. A tubing assembly is provided that is operably attached to the mouthpiece and the gas delivery pack for delivering gas from the gas delivery pack to the mouthpiece. A single-sided, hollow frame is provided. The frame at least partially encloses the mouthpiece and the tubing assembly and is adapted to attach to an ear, a hat, helmet or an eyeglass frame of a user. A bracket is provided. The bracket is adapted to support a pair of headphones for use with a portable music system. The bracket is attached to the frame and locates the headphones in positions suitable for mounting the headphones to ears of a user.

(8) In yet a further variant, the mouthpiece includes a switch for controlling flow of oxygen and air through the mouthpiece.

(9) In another variant of the invention, the at least one nose port is capable of forming a medical standard air-tight seal when inserted into a nostril.

(10) In still another variant, the mouth port is capable of forming a substantially air-tight seal when inserted into a mouth.

(11) In yet another variant, the tubing assembly includes a least one rotatable connector one of the headphones.

(12) In a further variant, the portable gas delivery system further includes at least one adapter that has a first recess and a second recess formed therein. The first recess is adapted to receive a portion of the tubing assembly and the second recess is adapted to receive a strap for mounting the tubing assembly with respect to a person's head.

(13) In yet a further variant, a portable gas delivery system includes a gas delivery pack that has a gas storage region, a gas compression region, a gas filtration region or a combination thereof. A garment is provided that has at least one pocket, the pocket is adapted to receive the gas delivery pack. A nosepiece is provided that has at least one nose port. A medical cannula tubing assembly is provided that is operably attached to the nosepiece, that conceals the medical nasal cannula, and the gas delivery pack for delivering a flow of gas from the gas delivery pack to the nosepiece. A single-sided, hollow frame is provided. The frame at least partially encloses the nosepiece, allowing the medical nasal cannula to protrude through the nosepiece for a snug fit into the nostrils forming a medical air-tight seal to the nose. The tubing assembly and is adapted to attach to an ear 66, a hat, helmet or an eyeglass frame of a user. A wireless transceiver adapted for use with a cellular telephone is provided. The transceiver has a listening portion and a speaking portion and is attached to the frame so as to position the listening portion adjacent a user's ear and the speaking portion adjacent a user's mouth.

(14) In still a further variant, at least one clip is provided. The clip is adapted to secure the medical cannula tubing assembly to the garment.

(15) In another variant of the invention, a portable gas delivery system includes a gas delivery pack that has a gas storage region, a gas compression region, a gas filtration region or a combination thereof. A garment is provided that has at least one pocket. The pocket is adapted to receive the gas delivery pack. A nosepiece is provided that has at least one nose port. A medical cannula tubing assembly is provided that is operably attached to the nosepiece and the gas delivery pack for delivering a flow of gas from the gas delivery pack to the nosepiece. A single-sided, hollow frame is provided. The frame at least partially encloses the nosepiece, allowing the medical nasal cannula to protrude through the nosepiece for a snug fit into the nostrils, and the tubing assembly and is adapted to attach to an ear 66, a hat, helmet or an eyeglass frame of a user. A bracket is provided. The bracket is adapted to support a pair of headphones for use with a portable music system. The bracket is attached to the frame and locates the headphones in positions suitable for mounting the headphones to ears of a user.

(16) In still another variant, at least one clip is provided. The clip is adapted to secure the medical cannula tubing assembly to the garment.

(17) In yet another variant, the garment further includes a second pocket. The second pocket is adapted to secure a portable music system for connection to the headphones.

(18) In a further variant, a portable gas delivery system includes a gas delivery pack that has a gas storage region, a gas compression region, a gas filtration region or a combination thereof. A headpiece is provided that includes a tubular arm that has a first end and a second end. A nosepiece is provided that is operably connected to the first end of the tubular arm. The nosepiece that conceals a medical nasal cannula has at least one nose port. A molded tube is provided that has a first and a second end. The first end is operably connected to the tubular arm. The molded tube is formed to mount to a person's body. A tubing assembly is provided that is operably attached to the second end of the tubular arm and the gas delivery pack. The gas delivery pack, tubing assembly and headpiece are in fluid communication for delivering a flow of gas from the gas delivery pack to the nosepiece. A wireless transceiver adapted for use with a cellular telephone is provided. The transceiver has a listening portion and a speaking portion and is attached to the tubular arm so as to position the listening portion adjacent a user's ear and the speaking portion adjacent a user's mouth.

(19) In still a further variant, the tubing assembly includes a flow switch operably connected to switch the flow of gas on or off.

(20) In yet a further variant, a cushion is attached to the molded tube.

(21) In another variant of the invention, the molded tube and the tubular arm are in fluid communication through a rotatable connector.

(22) In still another variant, the molded tube is adapted to mount to a human ear.

(23) In still another variant, the frame is designed to fold to fit a pocket and an elasticized string is connected to the frame and an interior surface of the pocket.

(24) In yet another variant, a portable gas delivery system includes a gas delivery pack that has a gas storage region, a gas compression region, a gas filtration region or a combination thereof. A headpiece is provided that includes a tubular arm that has a first end and a second end. A nosepiece operably connected to the first end of the tubular arm is provided. The nosepiece has at least one nose port. A molded tube is provided that has a first and a second end. The first end is operably connected to the tubular arm. The molded tube is formed to mount to a person's body. A medical cannula tubing assembly is provided that is operably attached to the second end of the tubular arm and the gas delivery pack. The gas delivery pack, tubing assembly and headpiece are in fluid communication for delivering a flow of gas from the gas delivery pack to the nosepiece. A bracket is provided. The bracket is adapted to support a pair of headphones for use with a portable music system. The bracket is attached to the molded tube and locates the headphones in positions suitable for mounting the headphones to ears of a user.

(25) In still another variant, the tubing assembly includes a flow switch operably connected to switch the flow of gas on or off.

(26) In a further variant, a cushion is attached to the molded tube.

(27) In still a further variant, the molded tube and the tubular arm are in fluid communication through a rotatable connector.

(28) In yet a further variant, the molded tube is adapted to mount to a human ear.

(29) In another variant of the invention, the frame is designed to fold to fit a pocket and an elasticized string is connected to the frame and an interior surface of the pocket.

(30) In yet another variant, a portable gas delivery system includes a nosepiece and has at least one nose port. A gas delivery pack is provided that has a gas storage region, a gas compression region, a gas filtration region or a combination thereof. A medical cannula tubing assembly is provided that is operably attached to the nosepiece that conceals the medical nasal cannula and the gas delivery pack for delivering gas from the gas delivery pack to the nosepiece. A single-sided, hollow frame is provided. The frame at least partially encloses the nosepiece allowing the medical nasal cannula to protrude through the nosepiece for a snug fit into the nostrils and the tubing assembly and is adapted to attach to an ear, a hat or an eyeglass frame of a user. A wireless transceiver adapted for use with a cellular telephone is provided. The transceiver has a listening portion and a speaking portion and is attached to the frame so as to position the listening portion adjacent a user's ear and the speaking portion adjacent a user's mouth.

(31) In a further variant, a portable gas delivery system includes a nosepiece and has at least one nose port. A gas delivery pack is provided that has a gas storage region, a gas compression region, a gas filtration region or a combination thereof. A medical cannula tubing assembly is provided that is operably attached to the nosepiece that conceals the medical nasal cannula tubing and the gas delivery pack for delivering gas from the gas delivery pack to the nosepiece. A single-sided, hollow frame is provided. The frame at least partially encloses the nosepiece allowing the medical nasal cannula to protrude through the nosepiece for a snug fit into the nostrils and the tubing assembly and is adapted to attach to an ear, a hat or an eyeglass frame of a user. A bracket is provided. The bracket is adapted to support a pair of headphones for use with a portable music system. The bracket is attached to the frame and locates the headphones in positions suitable for mounting the headphones to ears of a user.

(32) In another variant, an air filtration mask is provided. The mask is removably attached to the hollow frame.

(33) In a yet another variant, an air filtration mask is provided. The mask is removably attached to the hollow frame.

(34) In still another variant, an accordion pleated section adjacent said nosepiece is provided. The pleated section provides adjustment of said nosepiece to said user's nostrils.

(35)) In a final variant, an accordion pleated section adjacent said nosepiece is provided. The pleated section provides adjustment of said nosepiece to said user's nostrils.

### BRIEF DESCRIPTION OF THE DRAWINGS

FIG. 1 is a perspective view of a personal oxygen and air delivery system having a mouthpiece with both nose and mouth breathing ports;

FIG. 2 is a perspective view of the FIG. 1 embodiment illustrating a wireless transceiver for a cellular telephone;

FIG. 3 is a close-up perspective view of the breathing ports illustrating a switch for controlling flow through the mouthpiece;

FIG. 4 is a close-up perspective view of an earpiece illustrating a swivel mounting;

FIG. 5 is a perspective view of a clip for mounting to the tubing assembly and a carrying strap for a gas delivery pack;

FIG. 6 is a perspective view of the FIG. 1 embodiment illustrating an integral personal music system;

FIG. 7 is a perspective view of the FIG. 2 embodiment having only a nose port and attached to a user;

FIG. 8 is a perspective view of the FIG. 6 embodiment having only a nose port and attached to a user;

FIG. 9 is a perspective view of the FIG. 7 embodiment including a padded frame;

FIG. 10 is a perspective view of the FIG. 8 embodiment including a padded frame;

FIG. 11 is a perspective view of the FIG. 7 embodiment including a foldable frame attached to a user's garment;

FIG. 12 is a perspective view of the FIG. 7 embodiment;

FIG. 13 is a perspective view of the FIG. 8 embodiment with stereo speakers;

FIG. 14 is a perspective view of another embodiment including a molded tube resting on the neck of the user and a wireless transceiver for a cellular telephone;

FIG. 15 is a perspective view of another embodiment including a molded tube resting on the neck of the user and an integral personal music system;

FIG. 16 is a perspective view of the FIG. 14 embodiment showing a padded section on the molded tube;

FIG. 17 is a perspective view of the FIG. 15 embodiment;

FIG. 18 a perspective view of the FIG. 7 embodiment including a face mask; and

FIG. 19 a perspective view of the FIG. 8 embodiment including a face mask.

### DETAILED DESCRIPTION OF THE PREFERRED EMBODIMENTS

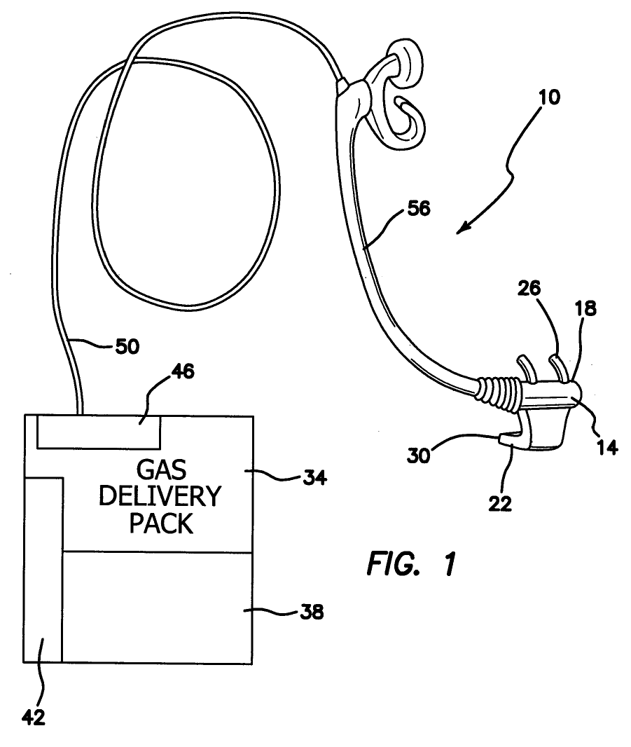

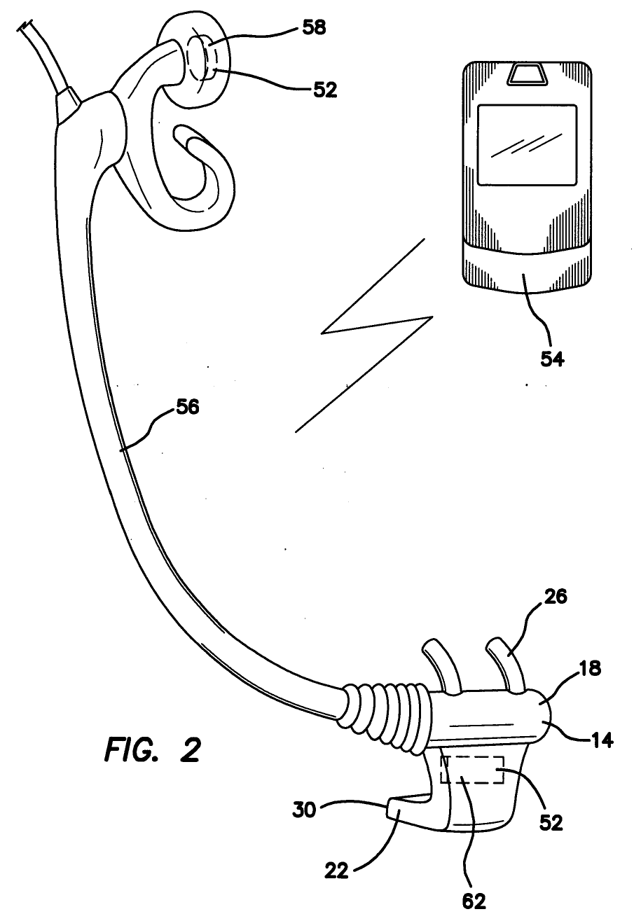

(1) FIGS. 1 and 2 illustrate a portable gas delivery system 10 that is constructed from the following components. A mouthpiece 14 is provided that has an upper region 18 and a lower region 22 extending from it. The upper region 18 includes at least one nose port 26 and the lower region 22 includes a mouth port 30. A gas delivery pack 34 is provided that has a gas storage region 38, a gas compression region 42, a gas filtration region 46 or a combination thereof. A medical cannula tubing assembly 50 is provided that is operably attached to the mouthpiece 14 and the gas delivery pack 34 for delivering gas from the gas delivery pack 24 to the mouthpiece 14. A single-sided, hollow frame 56 is provided. The frame 56 at least partially encloses the mouthpiece 14 and the tubing assembly 50 and is adapted to attach to an ear 66, a hat (not shown), helmet (not shown) or an eyeglass frame (not shown) of a user 134. A wireless transceiver 52 adapted for use with a cellular telephone 54 is provided. The transceiver 52 has a listening portion 58 and a speaking portion 62 and is attached to the frame 56 so as to position the listening portion 58 adjacent a user's ear 66 and the speaking portion 62 adjacent a user's mouth 70.

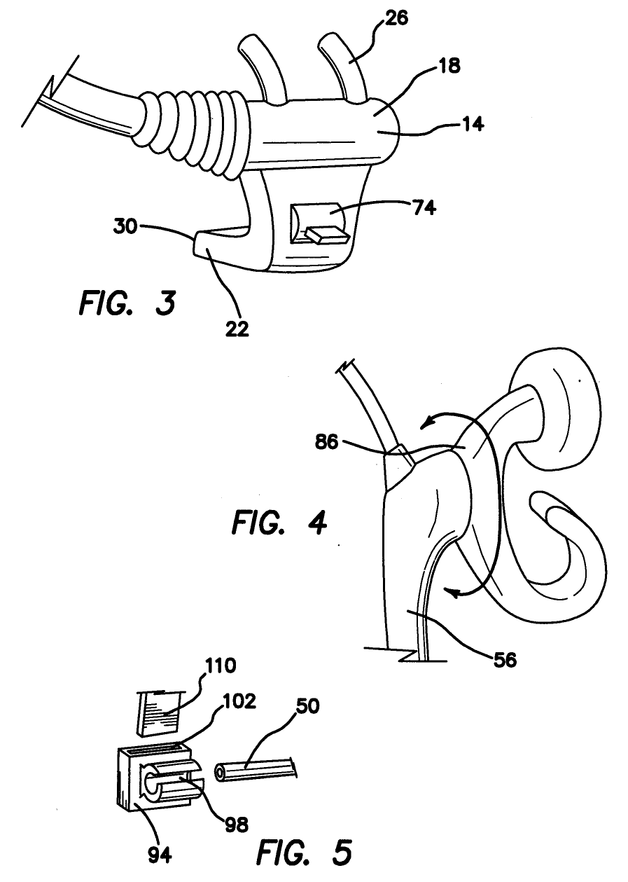

(2) In a variant of the invention, as illustrated in FIG. 3, the mouthpiece 14 includes a switch 74 for controlling flow of oxygen and air through the mouthpiece 14.

(3) In another variant, the at least one nose port 26 is capable of forming a medical standard air-tight seal when inserted into a nostril 78.

(4) In still another variant, the mouth port 30 is capable of forming a substantially air-tight seal when inserted into a mouth 70.

(5) In yet another variant, as illustrated in FIG. 4, the frame 56 includes a least one rotatable connector 86 adjacent the listening portion 58.

(6) In a further variant, as illustrated in FIG. 5, the portable gas delivery system includes at least one adapter 94 that has a first recess 98 and a second recess 102 formed in it. The first recess 98 is adapted to receive a portion 106 of the tubing assembly 50 and the second recess 102 is adapted to receive a strap 110 for mounting the tubing assembly 50 with respect to a person's head 114.

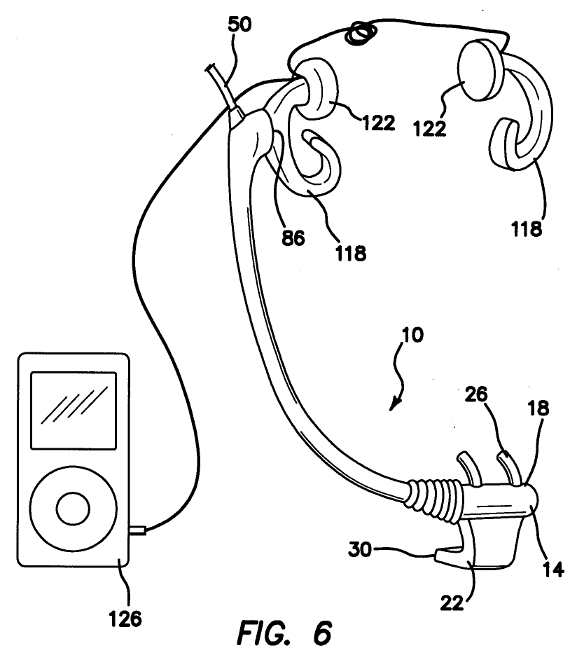

(7) In still a further variant, as illustrated in FIG. 6, a portable gas delivery system 10 includes a mouthpiece 14 that has an upper region 18 and a lower region 22 extending from it. The upper region 18 includes at least one nose port 26 and the lower region 22 includes a mouth port 30. A gas delivery pack 34 is provided that has a gas storage region 38, a gas compression region 42, a gas filtration region 46 or a combination thereof. A tubing assembly 50 is provided that is operably attached to the mouthpiece 14 and the gas delivery pack 34 for delivering gas from the gas delivery pack 34 to the mouthpiece 14. A single-sided, hollow frame 56 is provided. The frame 56 at least partially encloses the mouthpiece 14 and the tubing assembly 50 and is adapted to attach to an ear 66, a hat, helmet or an eyeglass frame of a user 134. A bracket 118 is provided. The bracket 118 is adapted to support a pair of headphones 122 for use with a portable music system 126. The bracket 118 is attached to the frame 56 and locates the headphones 122 in positions suitable for mounting the headphones 122 to ears 66 of a user 134.

(8) In yet a further variant, as illustrated in FIG. 3, the mouthpiece 14 includes a switch 74 for controlling flow of oxygen and air through the mouthpiece 14.

(9) In another variant of the invention, the at least one nose port 26 is capable of forming a medical standard air-tight seal when inserted into a nostril 78.

(10) In still another variant, the mouth port 30 is capable of forming a substantially air-tight seal when inserted into a mouth 70.

(11) In yet another variant, the tubing assembly 50 includes a least one rotatable connector 86 adjacent one of the headphones 122.

(12) In a further variant, as illustrated in FIG. 4, the portable gas delivery system includes at least one adapter 94 that has a first recess 98 and a second recess 102 formed in it. The first recess 98 is adapted to receive a portion 106 of the tubing assembly 50 and the second recess 102 is adapted to receive a strap 110 for mounting the tubing assembly 50 with respect to a person's head 114.

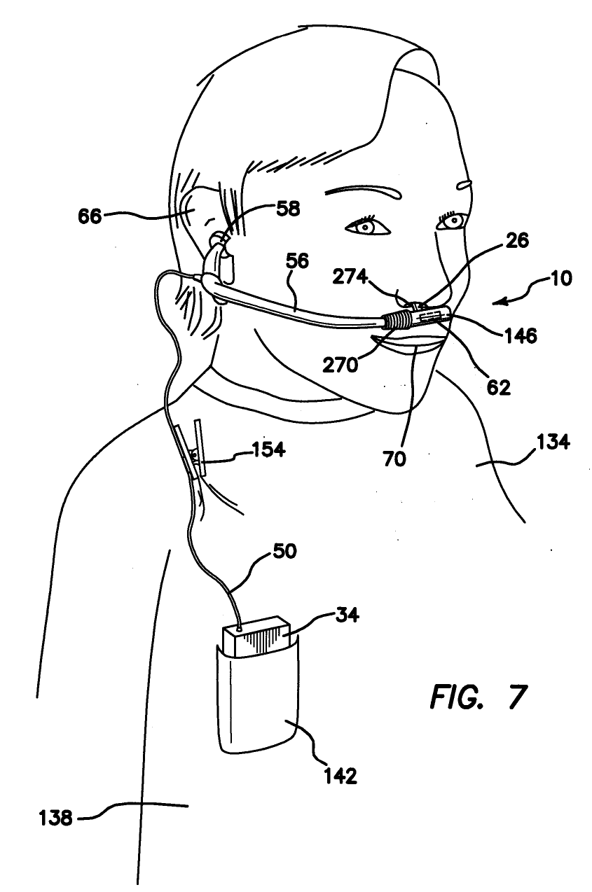

(13) In yet a further variant, as illustrated in FIG. 7, a portable gas delivery system 10 includes a gas delivery pack 34 that has a gas storage region 38, a gas compression region 42, a gas filtration region 46 or a combination thereof. A garment 138 is provided that has at least one pocket 142, the pocket 142 is adapted to receive the gas delivery pack 34. A nosepiece 146 is provided that has at least one nose port 26. A medical cannula tubing assembly 50 is provided that is operably attached to the nosepiece, that conceals the medical nasal cannula 146, and the gas delivery pack 34 for delivering a flow of gas from the gas delivery pack 34 to the nosepiece 146. A single-sided, hollow frame 56 is provided. The frame 56 at least partially encloses the nosepiece 146, allowing the medical nasal cannula 50 to protrude through the nosepiece 146, for a snug fit into the nostrils 274. The tubing assembly 50 and is adapted to attach to an ear 66, a hat, helmet or an eyeglass frame of a user 134. A wireless transceiver adapted for use with a cellular telephone 54 is provided. The transceiver 54 has a listening portion 58 and a speaking portion 62 and is attached to the frame 56 so as to position the listening portion 58 adjacent a user's ear 66 and the speaking portion 62 adjacent a user's mouth 70.

(14) In still a further variant, at least one clip 154 is provided. The clip 154 is adapted to secure the medical cannula tubing assembly 50 to the garment 138.

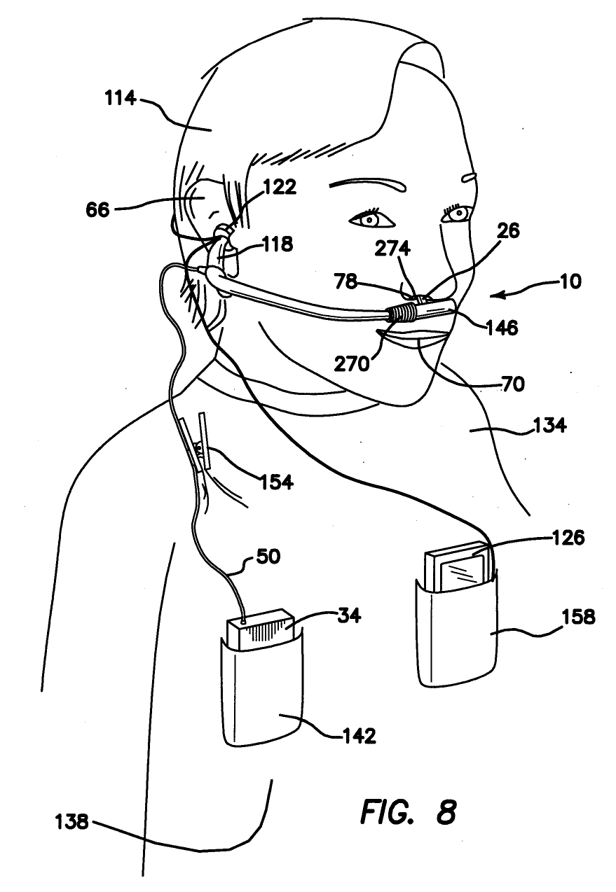

(15) In another variant of the invention, as illustrated in FIG. 8, a portable gas delivery system 10 includes a gas delivery pack 34 that has a gas storage region 38, a gas compression region 42, a gas filtration region 46 or a combination thereof. A garment 138 is provided that has at least one pocket 142. The pocket 142 is adapted to receive the gas delivery pack 34. A nosepiece 146 is provided that has at least one nose port 26. A medical cannula tubing assembly 50 is provided that is operably attached to the nosepiece 146 and the gas delivery pack 34 for delivering a flow of gas from the gas delivery pack 34 to the nosepiece 146. A single-sided, hollow frame 56 is provided. The frame 56 at least partially encloses the nosepiece, allowing the medical nasal cannula 50 to protrude through the nosepiece 146 for a snug fit into the nostrils 274, and the tubing assembly 50 and is adapted to attach to an ear 66, a hat, helmet or an eyeglass frame of a user 134. A bracket 118 is provided. The bracket 118 is adapted to support a pair of headphones 122 for use with a portable music system 126. The bracket 118 is attached to the frame 56 and locates the headphones 122 in positions suitable for mounting the headphones 122 to ears 66 of a user 134.

(16) In still another variant, at least one clip 154 is provided. The clip 154 is adapted to secure the medical cannula tubing assembly 50 to the garment 138.

(17) In yet another variant, the garment 138 further includes a second pocket 158. The second pocket 158 is adapted to secure a portable music system 126 for connection to the headphones 122.

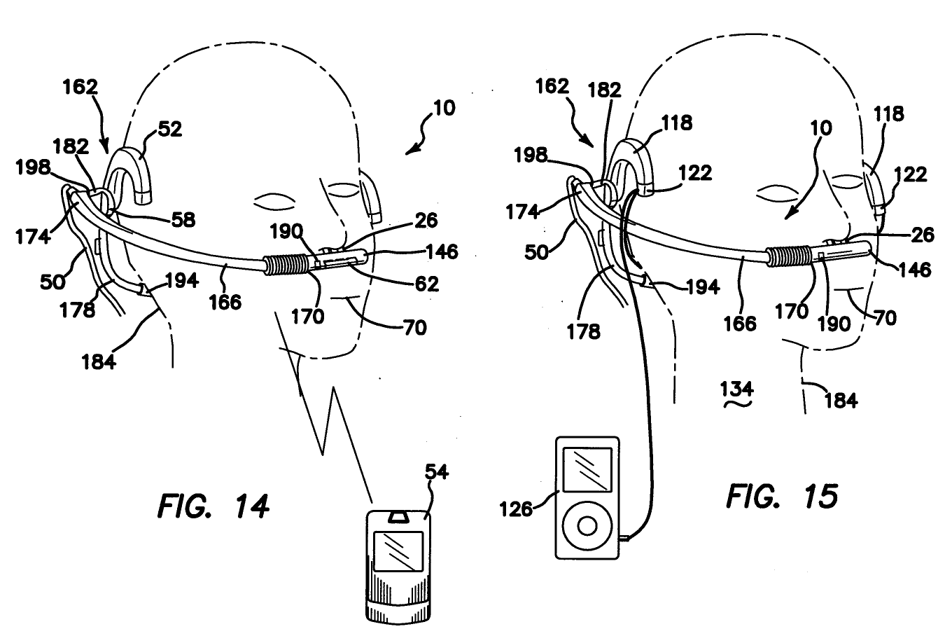

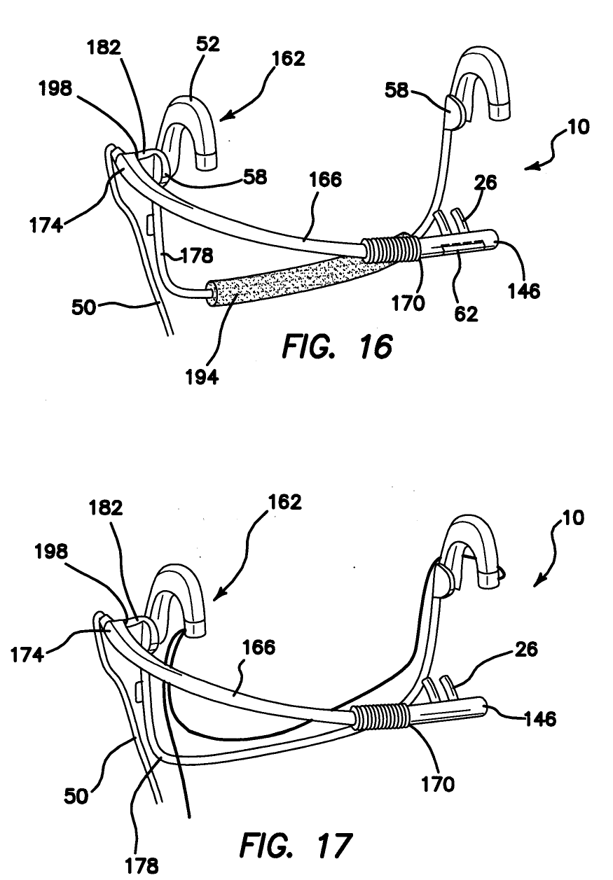

(18) In a further variant, as illustrated in FIGS. 14 and 16, a portable gas delivery system 10 includes a gas delivery pack 34 that has a gas storage region 38, a gas compression region 42, a gas filtration region 46 or a combination thereof. A headpiece 162 is provided that includes a tubular arm 166 that has a first end 170 and a second end 174. A nosepiece 146 is provided that is operably connected to the first end 170 of the tubular arm 166. The nosepiece 146 that conceals a medical nasal cannula has at least one nose port 26. A molded tube 178 is provided that has a first 182 and a second 186 end. The first end 182 is operably connected to the tubular arm 166. The molded tube 178 is formed to mount to a person's body 184. A tubing assembly 50 is provided that is operably attached to the second end 174 of the tubular arm 166 and the gas delivery pack 34. The gas delivery pack 34, tubing assembly 50 and headpiece 162 are in fluid communication for delivering a flow of gas from the gas delivery pack 34 to the nosepiece 146. A wireless transceiver 52 adapted for use with a cellular telephone 54 is provided. The transceiver 52 has a listening portion 58 and a speaking portion 62 and is attached to the tubular arm 166 so as to position the listening portion 58 adjacent a user's ear 66 and the speaking portion adjacent a user's mouth 70.

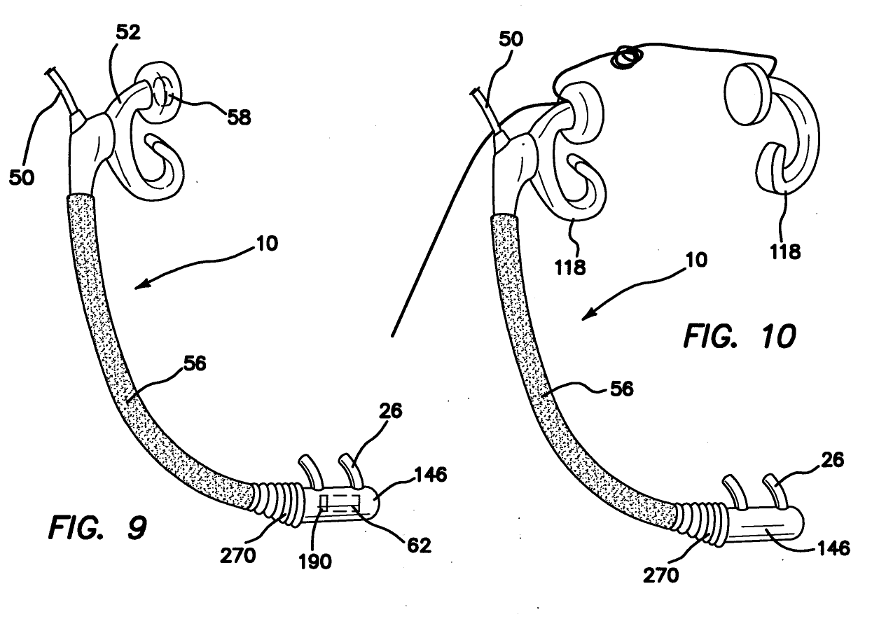

(19) In still a further variant, as illustrated in FIGS. 9, 12 and 14, the tubing assembly 50 includes a flow switch 190 operably connected to switch the flow of gas on or off.

(20) In yet a further variant, as illustrated in FIGS. 14 and 16, a cushion 194 is attached to the molded tube 178.

(21) In another variant of the invention, the molded tube 178 and the tubular arm 166 are in fluid communication through a rotatable connector 198.

(22) In still another variant, the molded tube 178 is adapted to mount to a human ear 66.

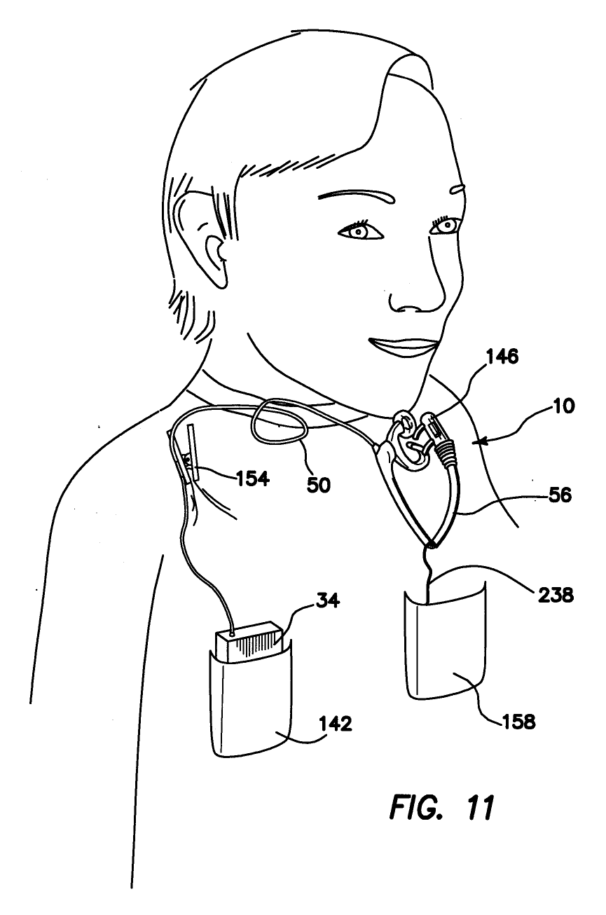

(23) In still another variant, as illustrated in FIG. 11, the frame 56 is designed to fold to fit a pocket 158 and an elasticized string 238 is connected to the frame 56 and an interior surface 234 of the pocket 158.

(24) In yet another variant, as illustrated in FIGS. 15 and 17, a portable gas delivery system 10 includes a gas delivery pack 34 that has a gas storage region 38, a gas compression region 42, a gas filtration region 46 or a combination thereof. A headpiece 162 is provided that includes a tubular arm 166 that has a first end 170 and a second end 174. A nosepiece 146 that conceals a medical nasal cannula is provided that is operably connected to the first end 170 of the tubular arm 166. The nosepiece 146 has at least one nose port 26. A molded tube 178 is provided that has a first 182 and a second 186 end. The first end 182 is operably connected to the tubular arm 166. The molded tube 178 is formed to mount to a person's body 184. A medical cannula tubing assembly 50 is provided that is operably attached to the second end 174 of the tubular arm 166 and the gas delivery pack 34. The gas delivery pack 34, medical cannula tubing assembly 50 and headpiece 162 are in fluid communication for delivering a flow of gas from the gas delivery pack 34 to the nosepiece 146, allowing the medical nasal cannula 50 to protrude through the nosepiece 146 for a snug fit into the nostrils 274, forming a medical air-tight seal of oxygen to the nostrils 274. A bracket 118 is provided. The bracket 118 is adapted to support a pair of headphones 122 for use with a portable music system 126. The bracket 118 is attached to the molded tube 178 and locates the headphones 122 in positions suitable for mounting the headphones 122 to ears 66 of a user 134.

(25) In still another variant, the tubing assembly 50 includes a flow switch 190 operably connected to switch the flow of gas on or off.

(26) In a further variant, a cushion 194 is attached to the molded tube.

(27) In still a further variant, the molded tube 178 and the tubular arm 166 are in fluid communication through a rotatable connector 198.

(28) In yet a further variant, the molded tube 178 is adapted to mount to a human ear 66.

(29) In still another variant, as illustrated in FIG. 11, the frame 56 is designed to fold to fit a pocket 158 and an elasticized string 238 is connected to the frame 56 and an interior surface 234 of the pocket 158.

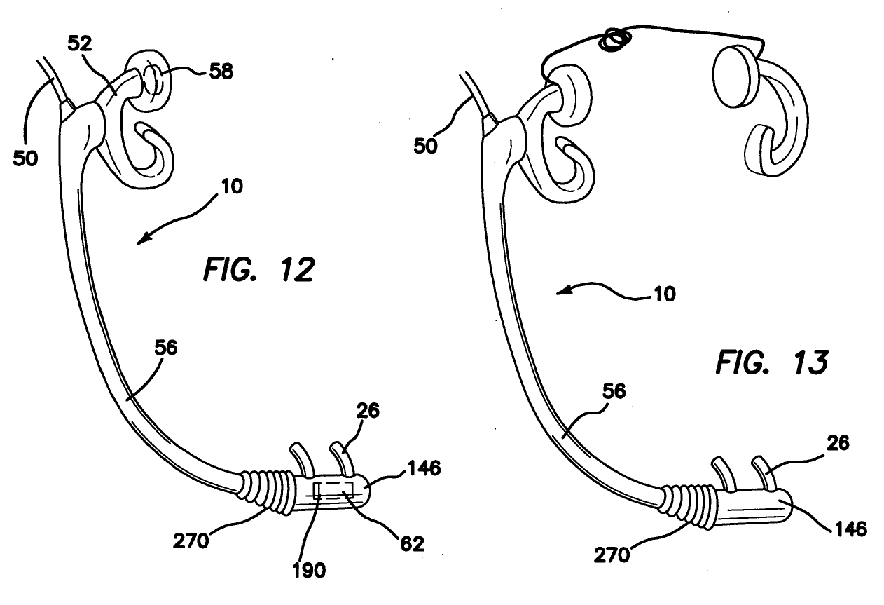

(30) In yet another variant, as illustrated in FIGS. 9 and 12, a portable gas delivery system 10 includes a nosepiece 146 that has at least one nose port 26. A gas delivery pack 34 is provided that has a gas storage region 38, a gas compression region 42, a gas filtration region 46 or a combination thereof. A medical cannula tubing assembly 50 is provided that is operably attached to the nosepiece 146 that conceals the medical nasal cannula tubing 50 and the gas delivery pack 34 for delivering gas from the gas delivery pack 34 to the nosepiece 146. A single-sided, hollow frame 56 is provided. The frame 56 at least partially encloses the nosepiece, allowing the medical nasal cannula to protrude through the nosepiece 146 for a snug fit into the nostrils, and the tubing assembly 50 and is adapted to attach to an ear 66, a hat, helmet or an eyeglass frame of a user 134. A wireless transceiver 52 adapted for use with a cellular telephone 54 is provided. The transceiver 52 has a listening portion 58 and a speaking portion 62 and is attached to the frame 56 so as to position the listening portion 58 adjacent a user's ear 66 and the speaking portion adjacent a user's mouth 70.

(31) In a further variant, as illustrated in FIGS. 10 and 13, a portable gas delivery system 10 includes a nosepiece 146 that has at least one nose port 26. A gas delivery pack 34 is provided that has a gas storage region 38, a gas compression region 42, a gas filtration region 46 or a combination thereof. A medical cannula tubing assembly 50 is provided that is operably attached to the nosepiece 146 that conceals the medical nasal cannula tubing 50, and the gas delivery pack 34 for delivering gas from the gas delivery pack 34 to the nosepiece 146. A single-sided, hollow frame 56 is provided. The frame 56 at least partially encloses the nosepiece allowing the medical nasal cannula to protrude through the nosepiece 146 for a snug fit into the nostrils 274 and the tubing assembly 50 and is adapted to attach to an ear 66, a hat, helmet or an eyeglass frame of a user. A bracket 118 is provided. The bracket 118 is adapted to support a pair of headphones 122 for use with a portable music system 126. The bracket 118 is attached to the frame 56 and locates the headphones 122 in positions suitable for mounting the headphones 122 to ears 66 of a user 134.

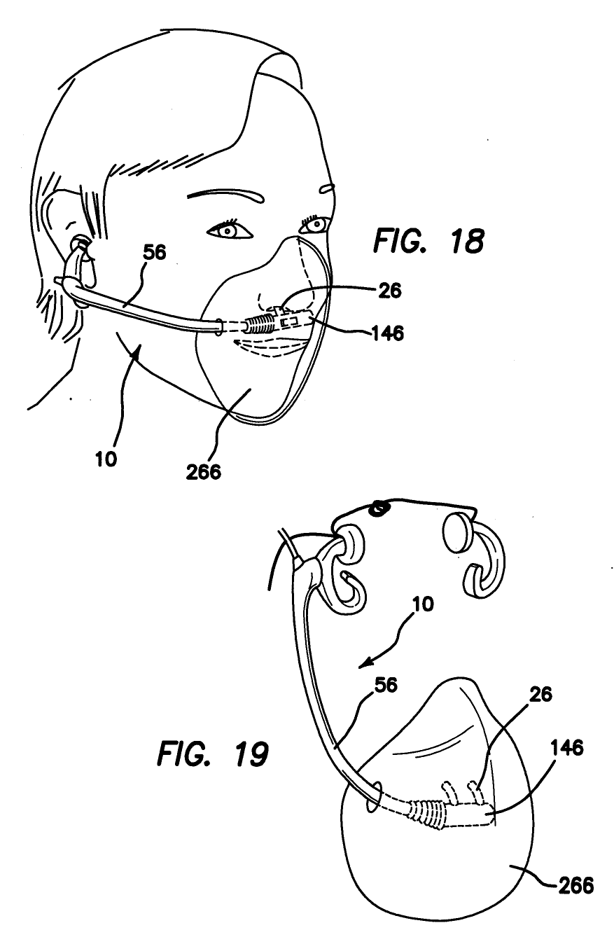

(32) In another variant, as illustrated in FIGS. 18 and 19, an air filtration mask 266 is provided. The mask 266 is removably attached to the hollow frame 56.

(33) In still another variant, an air filtration mask 266 is provided. The mask 266 is removably attached to the hollow frame 56.

(34) In still another variant, as illustrated in FIGS. 7, 9 and 12, an accordion pleated section 270 adjacent said nosepiece 146 is provided. The pleated section 270 provides adjustment of said nosepiece 146 to said user's nostrils 274.

(35)) In a final variant, an accordion pleated section 270 as illustrated in FIGS. 8, 10 and 13, adjacent said nosepiece 146 is provided. The pleated section 270 provides adjustment of said nosepiece 146 to said user's nostrils 274.

It is contemplated that features disclosed in this application, as well as those described in the above applications incorporated by reference, can be mixed and matched to suit particular circumstances. Various other modifications and changes will be apparent to those of ordinary skill.

1. A portable gas delivery system comprising:
a mouthpiece having an upper region and a lower region extending therefrom, wherein the upper region includes at least one nose port and wherein the lower region includes a mouth port;
a gas delivery pack having a gas storage region, a gas compression region, a gas filtration region or a combination thereof;
a tubing assembly that is operably attached to the mouthpiece and the gas delivery pack for delivering gas from the gas delivery pack to the mouthpiece;
a single-sided, hollow frame, said frame at least partially enclosing said mouthpiece and said tubing assembly and being adapted to attach to an ear, a hat, helmet or an eyeglass frame of a user; and
a wireless transceiver adapted for use with a cellular telephone, said transceiver having a listening portion and a speaking portion and being attached to said frame so as to position said listening portion adjacent a user's ear and said speaking portion adjacent a user's mouth.

2. The portable gas delivery system of claim 1, wherein the mouthpiece further comprises a switch for controlling flow of oxygen and air through the mouthpiece.

3. The portable gas delivery system of claim 1, wherein the at least one nose port is capable of forming a medical standard air-tight seal when inserted into a nostril.

4. The portable gas delivery system of claim 1, wherein the mouth port is capable of forming a substantially air-tight seal when inserted into a mouth.

5. The portable gas delivery system of claim 1, wherein the tubing assembly includes a least one rotatable connector adjacent said listening portion.

6. The portable gas delivery system of claim 1, further comprising at least one adapter having a first recess and a second recess formed therein, wherein the first recess is adapted to receive a portion of the tubing assembly and wherein the second recess is adapted to receive a strap for mounting the tubing assembly with respect to a person's head.

7. A portable gas delivery system comprising:
a mouthpiece having an upper region and a lower region extending therefrom, wherein the upper region includes at least one nose port and wherein the lower region includes a mouth port;
a gas delivery pack having a gas storage region, a gas compression region, a gas filtration region or a combination thereof;
a tubing assembly that is operably attached to the mouthpiece and the gas delivery pack for delivering gas from the gas delivery pack to the mouthpiece;
a single-sided, hollow frame, said frame at least partially enclosing said mouthpiece and said tubing assembly and being adapted to attach to an ear, a hat, helmet or an eyeglass frame of a user; and
a bracket, said bracket adapted to support a pair of headphones for use with a portable music system, said bracket being attached to said frame and disposing said headphones in positions suitable for mounting said headphones to ears of a user.

8. The portable gas delivery system of claim 7, wherein the mouthpiece further comprises a switch for controlling flow of oxygen and air through the mouthpiece.

9. The portable gas delivery system of claim 7, wherein the at least one nose port is capable of forming a medical standard air-tight seal when inserted into a nostril.

10. The portable gas delivery system of claim 7, wherein the mouth port is capable of forming a substantially air-tight seal when inserted into a mouth.

11. The portable gas delivery system of claim 7, wherein the tubing assembly includes a least one rotatable connector one of said headphones.

12. The portable gas delivery system of claim 7, further comprising at least one adapter having a first recess and a second recess formed therein, wherein the first recess is adapted to receive a portion of the tubing assembly and wherein the second recess is adapted to receive a strap for mounting the tubing assembly with respect to a person's head.

13. A portable gas delivery system comprising:
a gas delivery pack having a gas storage region, a gas compression region, a gas filtration region or a combination thereof;
a garment having at least one pocket, the pocket being adapted to receive the gas delivery pack;
a nosepiece having at least one nose port;
a medical cannula tubing assembly that is operably attached to the nosepiece, that conceals said medical nasal cannula, and the gas delivery pack for delivering a flow of gas from the gas delivery pack to the nosepiece;
a single-sided, hollow frame, said frame at least partially enclosing said nosepiece and said tubing assembly and being adapted to attach to an ear, a hat, helmet or an eyeglass frame of a user; and
a wireless transceiver adapted for use with a cellular telephone, said transceiver having a listening portion and a speaking portion and being attached to said frame so as to position said listening portion adjacent a user's ear and said speaking portion adjacent a user's mouth.

14. The portable gas delivery system of claim 13 further comprising at least one clip, said clip being adapted to secure said tubing assembly to said garment.

15. A portable gas delivery system comprising:
a gas delivery pack having a gas storage region, a gas compression region, a gas filtration region or a combination thereof;
a garment having at least one pocket, the pocket being adapted to receive the gas delivery pack;
a nosepiece having at least one nose port;
a medical cannula tubing assembly that is operably attached to the nosepiece that conceals said medical nasal cannula and the gas delivery pack for delivering a flow of gas from the gas delivery pack to the nosepiece;
a single-sided, hollow frame, said frame at least partially enclosing said nosepiece and said tubing assembly and being adapted to attach to an ear, a hat, helmet or an eyeglass frame of a user; and
a bracket, said bracket adapted to support a pair of headphones for use with a portable music system, said bracket being attached to said frame and disposing said headphones in positions suitable for mounting said headphones to ears of a user.

16. The portable gas delivery system of claim 15 further comprising at least one clip, said clip being adapted to secure said tubing assembly to said garment.

17. The portable gas delivery system of claim 16, wherein said garment further comprises a second pocket, said second pocket adapted to secure a portable music system for connection to said headphones.

18. A portable gas delivery system comprising:
a gas delivery pack having a gas storage region, a gas compression region, a gas filtration region or a combination thereof;
a headpiece comprising: a tubular arm having a first end and a second end;
a nosepiece operably connected to the first end of the tubular arm, the nosepiece having at least one nose port;
a molded tube having a first and a second end, the first end operably connected to the tubular arm, the molded tube formed to mount to a person's body;
a tubing assembly that is operably attached to the second end of the tubular arm and the gas delivery pack;
the gas delivery pack, tubing assembly and headpiece being in fluid communication for delivering a flow of gas from the gas delivery pack to the nosepiece; and
a wireless transceiver adapted for use with a cellular telephone, said transceiver having a listening portion and a speaking portion and being attached to said tubular arm so as to position said listening portion adjacent a user's ear and said speaking portion adjacent a user's mouth.

19. The portable gas delivery system of claim 18 wherein the tubing assembly includes a flow switch operably connected to switch the flow of gas on or off.

20. The portable gas delivery system of claim 18 wherein a cushion is attached to the molded tube.

21. The portable gas delivery system of claim 18 wherein the molded tube and the tubular arm are in fluid communication through a rotatable connector.

22. The portable gas delivery system of claim 18 wherein the molded tube is adapted to mount to a human ear.

23. The portable gas delivery system of claim 18 wherein said frame is designed to fold to fit a pocket and an elasticized string is connected to the frame and an interior surface of said pocket.

24. A portable gas delivery system comprising:
a gas delivery pack having a gas storage region, a gas compression region, a gas filtration region or a combination thereof;
a headpiece comprising: a tubular arm having a first end and a second end;
a nosepiece operably connected to the first end of the tubular arm, the nosepiece having at least one nose port;
a molded tube having a first and a second end, the first end operably connected to the tubular arm, the molded tube formed to mount to a person's body;
a medical cannula tubing assembly that is operably attached to the second end of the tubing assembly and the gas delivery pack;
the gas delivery pack, tubing assembly and headpiece being in fluid communication for delivering a flow of gas from the gas delivery pack to the nosepiece; and
a bracket, said bracket adapted to support a pair of headphones for use with a portable music system, said bracket being attached to said molded tube and disposing said headphones in positions suitable for mounting said headphones to ears of a user.

25. The portable gas delivery system of claim 24 wherein the tubing assembly includes a flow switch operably connected to switch the flow of gas on or off.

26. The portable gas delivery system of claim 24 wherein a cushion is attached to the molded tube.

27. The portable gas delivery system of claim 24 wherein the molded tube and the tubular arm are in fluid communication through a rotatable connector.

28. The portable gas delivery system of claim 24 wherein the molded tube is adapted to mount to a human ear.

29. The portable gas delivery system of claim 24 wherein said frame is designed to fold to fit a pocket and an elasticized string is connected to the frame and an interior surface of said pocket.

30. A portable gas delivery system comprising:
a nosepiece having at least one nose port;
a gas delivery pack having a gas storage region, a gas compression region, a gas filtration region or a combination thereof;
a medical cannula tubing assembly that is operably attached to said nosepiece, said nosepiece concealing said cannula tubing, and the gas delivery pack for delivering gas from the gas delivery pack to the nosepiece;
a single-sided, hollow frame, said frame at least partially enclosing said nosepiece, allowing said medical nasal cannula to protrude through said nosepiece for a snug fit into nostrils of a user, forming a medical air-tight seal of oxygen to said nostrils, and said tubing assembly and being adapted to attach to an ear, a hat, a helmet or an eyeglass frame of a user;
a wireless transceiver adapted for use with a cellular telephone, said transceiver having a listening portion and a speaking portion and being attached to said frame so as to position said listening portion adjacent a user's ear and said speaking portion adjacent a user's mouth.

31. A portable gas delivery system comprising:
a nosepiece having at least one nose port;
a gas delivery pack having a gas storage region, a gas compression region, a gas filtration region or a combination thereof;
a medical cannula tubing assembly that is operably attached to said nosepiece, said nosepiece concealing said medical nasal cannula, and the gas delivery pack for delivering gas from the gas delivery pack to the nosepiece;
a single-sided, hollow frame, said frame at least partially enclosing said nosepiece, allowing said medical nasal cannula to protrude through said nosepiece for a snug fit into nostrils of a user, forming a medical air-tight seal of oxygen to said nostrils, and said tubing assembly and being adapted to attach to an ear, a hat, a helmet or an eyeglass frame of a user;
a bracket, said bracket adapted to support a pair of headphones for use with a portable music system, said bracket being attached to said frame and disposing said headphones in positions suitable for mounting said headphones to ears of a user.

32. The portable gas delivery system, of claim 30, further comprising an air filtration mask, said mask removably attached to said hollow frame.

33. The portable gas delivery system, of claim 31, further comprising an air filtration mask, said mask removably attached to said hollow frame.

34. The portable gas delivery system, of claim 30, further comprising an accordion pleated section adjacent said nosepiece, said section providing adjustment of said nosepiece to said user's nostrils.

35. The portable gas delivery system, of claim 31, further comprising an accordion pleated section adjacent said nosepiece, said section providing adjustment of said nosepiece to said user's nostrils.
